(19) United States
(12) Patent Application Publication
(10) Pub. No.: US 2025/0116623 A1
(43) Pub. Date: Apr. 10, 2025

(54) HYDROGEN DETECTION DEVICE AND METHOD FOR MANUFACTURING THE SAME

(71) Applicant: Nuvoton Technology Corporation Japan, Kyoto (JP)

(72) Inventors: Kazunari HOMMA, Gifu (JP); Satoru ITO, Hyogo (JP); Ken KAWAI, Nara (JP); Koji KATAYAMA, Nara (JP)

(21) Appl. No.: 18/990,893

(22) Filed: Dec. 20, 2024

Related U.S. Application Data
(63) Continuation of application No. PCT/JP2023/024224, filed on Jun. 29, 2023.

(30) Foreign Application Priority Data
Jul. 4, 2022 (JP) 2022-107765

(51) Int. Cl. G01N 27/04; G01N 33/00

(52) U.S. Cl. CPC G01N 27/045; G01N 33/005

(57) ABSTRACT

A hydrogen detection device includes a bridge circuit including: a hydrogen sensor that is a first resistive element; a resistor that is a second resistive element; a reference element that is a third resistive element; and a resistor that is a fourth resistive element, and at least the hydrogen sensor and the reference element are provided on a single semiconductor chip. The hydrogen sensor includes a first electrode, a second electrode, a metal oxide layer, an insulating film, or the like, and the insulating film includes an opening through which part of the second electrode is exposed. The reference element includes a first electrode, a second electrode, a metal oxide layer, an insulating film, or the like, and the insulating film includes no opening through which part of the second electrode is exposed.

### CROSS REFERENCE TO RELATED APPLICATION(S)

This is a continuation application of PCT International Application No. PCT/JP2023/024224 filed on Jun. 29, 2023, designating the United States of America, which is based on and claims priority of Japanese Patent Application No. 2022-107765 filed on Jul. 4, 2022. The entire disclosures of the above-identified applications, including the specifications, drawings and claims are incorporated herein by reference in their entirety.

### FIELD

The present disclosure relates to a hydrogen detection device and a manufacturing method thereof, and in particular relates to: a hydrogen detection device including a bridge circuit; and a manufacturing method thereof.

### BACKGROUND

A hydrogen detection device that includes a bridge circuit including four resistive elements has been conventionally proposed (see Patent Literature (PTL) 1, for example). It should be noted that the bridge circuit is a Wheatstone bridge circuit.

### CITATION LIST

### Patent Literature

PTL 1: Japanese Unexamined Patent Application Publication No. 2019-152451

### SUMMARY

### Technical Problem

However, the hydrogen detection device disclosed in PTL 1 requires a heater and a temperature controller, and therefore needs to be improved upon.

In view of the above, the present disclosure provides: a hydrogen detection device that includes a bridge circuit, does not necessarily require a heater, and can operate stably; and a manufacturing method thereof.

### Solution to Problem

A hydrogen detection device according to an aspect of the present disclosure includes: a bridge circuit including a first resistive element, a second resistive element, a third resistive element, and a fourth resistive element. One end of the first resistive element and one end of the second resistive element are connected to each other, one end of the third resistive element and one end of the fourth resistive element are connected to each other, an other end of the first resistive element and an other end of the third resistive element are connected to each other, and an other end of the second resistive element and an other end of the fourth resistive element are connected to each other. Among the first resistive element, the second resistive element, the third resistive element, and the fourth resistive element, at least the first resistive element and the third resistive element are provided on a single semiconductor chip. The first resistive element is a hydrogen sensor and includes: a first electrode including a principal surface and a second electrode including a principal surface, the principal surface of the first electrode and the principal surface of the second electrode facing each other; a first metal oxide layer disposed in contact with the principal surface of the first electrode and the principal surface of the second electrode; and a first insulating film that covers the first electrode, the second electrode, and the first metal oxide layer. The first insulating film includes a first opening that is not covered by the first insulating film and through which part of an other surface of the second electrode opposite to the principal surface of the second electrode is exposed. The third resistive element is a reference element and includes: a third electrode including a principal surface and a fourth electrode including a principal surface, the principal surface of the third electrode and the principal surface of the fourth electrode facing each other; a second metal oxide layer disposed in contact with the principal surface of the third electrode and the principal surface of the fourth electrode; and a second insulating film that covers the third electrode, the fourth electrode, and the second metal oxide layer. The second insulating film includes no opening that is not covered by the second insulating film and through which part of an other surface of the fourth electrode opposite to the principal surface of the fourth electrode is exposed.

A manufacturing method for manufacturing a hydrogen detection device according to an aspect of the present disclosure is a manufacturing method for manufacturing a hydrogen detection device that includes a bridge circuit including: a first resistive element that is a hydrogen sensor; a second resistive element; a third resistive element that is a reference element; and a fourth resistive element. The manufacturing method includes: forming a layered structure for the first resistive element and the third resistive element; and forming an opening in the layered structure formed. In the forming of a layered structure, a layered structure including: a first electrode including a principal surface and a second electrode including a principal surface, the principal surface of the first electrode and the principal surface of the second electrode facing each other; a metal oxide layer disposed in contact with the principal surface of the first electrode and the principal surface of the second electrode; and an insulating film that covers the first electrode, the second electrode, and the metal oxide layer is formed as the layered structure for the first resistive element and the third resistive element. In the forming of an opening, at least a first opening that is not covered by the insulating film and through which part of an other surface of the second electrode opposite to the principal surface of the second electrode is exposed is formed in the insulating film of a portion of the layered structure, the portion corresponding to the first resistive element.

### Advantageous Effects

The present disclosure provides: a hydrogen detection device that includes a bridge circuit, does not necessarily require a heater, and can operate stably; and a manufacturing method thereof.

### BRIEF DESCRIPTION OF DRAWINGS

These and other advantages and features will become apparent from the following description thereof taken in conjunction with the accompanying Drawings, by way of non-limiting examples of embodiments disclosed herein.

FIG. 1 is an equivalent circuit diagram of a hydrogen detection device according to an embodiment.

FIG. 2A is a cross-sectional view illustrating a configuration example of a hydrogen sensor illustrated in FIG. 1.

FIG. 2B is a top view illustrating the configuration example of the hydrogen sensor illustrated in FIG. 2A.

FIG. 3 is a cross-sectional view illustrating a configuration example of a reference element illustrated in FIG. 1.

FIG. 4A is a schematic view of a configuration example of the hydrogen detection device according to the embodiment as a whole.

FIG. 4B is a plan view illustrating an example of a layout of wiring patterns of the hydrogen sensor and the reference element in the hydrogen detection device illustrated in FIG. 4A.

FIG. 5 is a flowchart illustrating a manufacturing method for manufacturing the hydrogen detection device according to the embodiment.

FIG. 6 is a diagram illustrating a result of an experiment regarding time dependency and distance dependency of an output voltage (differential voltage) of the hydrogen detection device according to the embodiment.

FIG. 7 is a diagram illustrating a result of an experiment regarding reaction, to hydrogen, of the hydrogen detection device according to the embodiment.

FIG. 8 is a schematic view of a configuration example of a hydrogen detection device according to Variation 1 of the embodiment as a whole.

FIG. 9A is a schematic view of a configuration example of a hydrogen detection device according to Variation 2 of the embodiment as a whole.

FIG. 9B is a plan view illustrating an example of a layout of wiring patterns of a hydrogen sensor and a reference element in the hydrogen detection device illustrated in FIG. 9A.

FIG. 10 is a flowchart illustrating a manufacturing method for manufacturing the hydrogen detection device according to Variation 2 of the embodiment.

FIG. 11 is a schematic view of a configuration example of a hydrogen detection device according to Variation 3 of the embodiment as a whole.

### DESCRIPTION OF EMBODIMENT

Hereinafter, an embodiment of the present disclosure is described in detail with reference to the Drawings. It should be noted that the embodiment described below shows a specific example of the present disclosure. The numerical values, shapes, materials, constituent elements, the arrangement and connection of the constituent elements, steps, the order of the steps, etc., in the following embodiment are mere examples, and therefore do not intend to limit the present disclosure. Moreover, each drawing is not necessarily an exact depiction. In each drawing, elements that are substantially the same share the same reference signs, and overlapping description thereof is omitted or simplified. Moreover, “A and B are connected to each other” means that A and B are electrically connected to each other, and includes not only a case in which A and B are directly connected to each other but also a case in which A and B are indirectly connected to each other in a state where another circuit element is interposed between A and B.

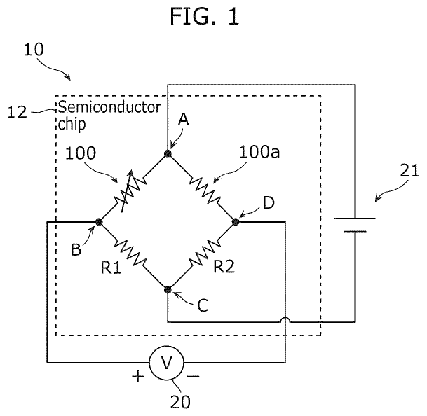

FIG. 1 is an equivalent circuit diagram of hydrogen detection device 10 according to the embodiment. In the present drawing, voltmeter 20 and DC voltage source 21 are also illustrated as external devices.

Hydrogen detection device 10 includes a bridge circuit including hydrogen sensor 100 that is a first resistive element, resistor R1 that is a second resistive element, reference element 100a that is a third resistive element, and resistor R2 that is a fourth resistive element. Each of one end of hydrogen sensor 100 and one end of resistor R1 is connected to terminal B, and each of one end of reference element 100a and one end of resistor R2 is connected to terminal D. Each of the other end of hydrogen sensor 100 and the other end of reference element 100a is connected to terminal A, and each of the other end of resistor R1 and the other end of resistor R2 is connected to terminal D. Hydrogen sensor 100, resistor R1, reference element 100a, and resistor R2 are provided on a single semiconductor chip 12.

In a hydrogen-free environment, hydrogen sensor 100 and reference element 100a have the same resistance value. In a hydrogen-containing environment, only the resistance value of hydrogen sensor 100 decreases according to the hydrogen concentration. Resistor R1 and resistor R2 have the same resistance value, and are each a resistor that has a fixed resistive value, such as 20Ω, and includes polysilicon or the like.

A voltage of terminal B based on terminal D is measured by voltmeter 20 in a state where DC voltage from DC voltage source 21 is applied between terminal A and terminal C of hydrogen detection device 10. Since the resistance value of hydrogen sensor 100 decreases according to the hydrogen concentration, the resistance balance in the bridge circuit is disrupted, a potential difference is generated between terminal B and terminal D, and the potential difference is measured by voltmeter 20.

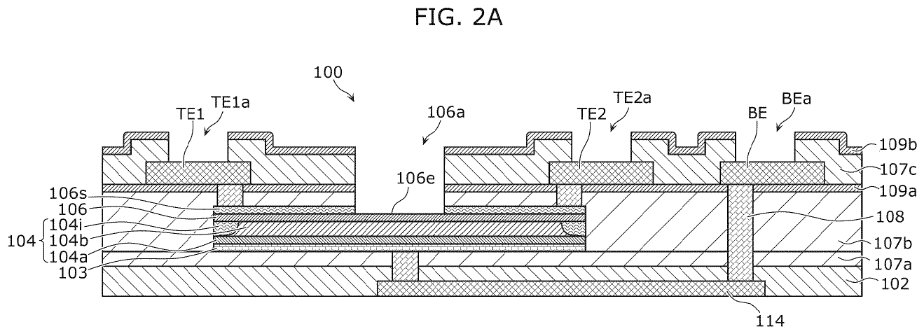

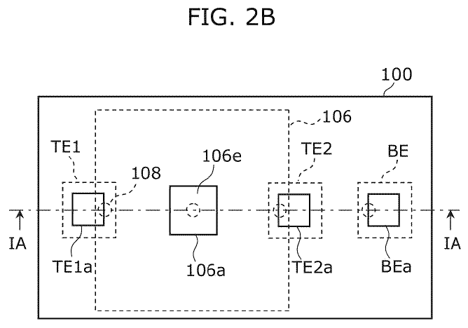

FIG. 2A is a cross-sectional view illustrating a configuration example of hydrogen sensor 100 illustrated in FIG. 1. FIG. 2B is a top view illustrating the configuration example of hydrogen sensor 100 illustrated in FIG. 2A. It should be noted that FIG. 2A schematically illustrates a cross section along line IA-IA in FIG. 2B viewed in the arrow direction.

Hydrogen sensor 100 includes, as main constituent elements: first electrode 103 including a principal surface and second electrode 106 including a principal surface, the principal surface of first electrode 103 and the principal surface of second electrode 106 facing each other; metal oxide layer 104 as a first metal oxide layer disposed in contact with the principal surface (i.e., upper surface) of first electrode 103 and the principal surface (i.e., lower surface) of second electrode 106; and a first insulating film (insulating films 107a to 107c, 109a, and 109b) that covers first electrode 103, second electrode 106, and metal oxide layer 104. The first insulating film includes opening 106a that is not covered by the first insulating film and through which part of the other surface (i.e., upper surface) of second electrode 106 opposite to the principal surface of second electrode 106 is exposed. In the present embodiment, metal layer 106s is also removed to expose second electrode 106 through opening 106a.

Hydrogen sensor 100 includes three terminals (first terminal TE1, second terminal TE2, and third terminal BE) for connection to the outside. Each of first terminal TE1 and second terminal TE2 is connected, through via 108, to the other surface of second electrode 106. Third terminal BE is connected, through wiring 114 and via 108, to the other surface (i.e., lower surface) of first electrode 103 opposite to the principal surface of first electrode 103. When hydrogen sensor 100 is used in a horizontal mode in which current flows in a horizontal direction in FIG. 2A to detect low-concentration hydrogen, first terminal TE1 and second terminal TE2 as one end and the other end of hydrogen sensor 100 are connected to other resistive elements. In contrast, when hydrogen sensor 100 is used in a vertical mode in which current flows in a vertical direction in FIG. 2A to detect high-concentration hydrogen, either first terminal TE1 or second terminal TE2 and third terminal BE as one end and the other end of hydrogen sensor 100 are connected to other resistive elements

It should be noted that although hydrogen sensor 100 illustrated in the present drawing can be used in both the horizontal mode and the vertical mode, the hydrogen sensor included in hydrogen detection device 10 is not limited to this example, and, for example, a hydrogen sensor that is dedicated to the horizontal mode and not provided with third terminal BE may be used.

The configuration, material, etc. of each constituent element are described in detail below.

First electrode 103 is a planar electrode and includes two surfaces. Of the two surfaces of first electrode 103, one surface (i.e., the upper surface in FIG. 2A) is in contact with metal oxide layer 104, and the other surface (i.e., the lower surface in FIG. 2A) is in contact with insulating film 107a and via 108. In FIG. 2B, first electrode 103 is in a rectangular shape of the same size as second electrode 106. First electrode 103 may be formed of, for example, a material having a standard electrode potential lower than that of metals included in metal oxides, such as tungsten, nickel, tantalum, titanium, aluminum, tantalum nitride, and titanium nitride. The higher the value of the standard electrode potential is, the more resistant to oxidation the material is. First electrode 103 in FIG. 2A is formed of, for example, transition metal nitride such as tantalum nitride (TaN) or titanium nitride (TiN), or a layered structure thereof.

Metal oxide layer 104 is disposed between the principal surface of first electrode 103 and the principal surface of second electrode 106 facing each other, includes a metal oxide serving as a gas-sensitive resistance film, and has a resistance value that reversibly changes according to the presence and absence of hydrogen-containing gas in gas in contact with second electrode 106. It is sufficient so long as metal oxide layer 104 has a property that enables its resistance to change according to hydrogen. Metal oxide layer 104 includes an oxygen-deficient metal oxide, for example. As the base metal of metal oxide layer 104, at least one of the following may be selected: aluminum (Al) and transition metals such as tantalum (Ta), hafnium (Hf), titanium (Ti), zirconium (Zr), niobium (Nb), tungsten (W), nickel (Ni), and iron (Fe).

Since transition metals can be in various oxidation states, it is possible to achieve different resistance states through redox reactions. Here, the “degree of oxygen deficiency” of a metal oxide is the percentage of the deficient amount of oxygen in the metal oxide relative to the amount of oxygen in a stoichiometric oxide composed of the same elements as the metal oxide. Here, the deficient amount of oxygen is a value obtained by subtracting the amount of oxygen in the metal oxide from the amount of oxygen in the stoichiometric metal oxide. When there are a plurality of stoichiometric metal oxides each composed of the same elements as the metal oxide, the degree of oxygen deficiency of the metal oxide is defined based on a stoichiometric metal oxide having the highest resistance value among the plurality of stoichiometric metal oxides. A stoichiometric metal oxide is more stable and has a higher resistance value than a non-stoichiometric metal oxide.

For example, when the base metal of metal oxide layer 104 is tantalum (Ta), a stoichiometric oxide as defined above is Ta₂O₅, and can be expressed as TaO_{2.5}. The degree of oxygen deficiency of TaO_{2.5} is 0%, and the degree of oxygen deficiency of TaO_{1.5} is obtained by the following equation: (2.5−1.5)/2.5=40%. Moreover, the degree of oxygen deficiency of a metal oxide having excess oxygen becomes a negative value. It should be noted that in the present disclosure, a degree of oxygen deficiency can be a positive value, 0, or a negative value, unless otherwise specified. An oxide having a low degree of oxygen deficiency has a high resistance value since it is closer to a stoichiometric oxide, whereas an oxide having a high degree of oxygen deficiency has a low resistance value since it is closer to a metal included in the oxide.

Metal oxide layer 104 illustrated in FIG. 2A includes: first layer 104a in contact with first electrode 103; second layer 104b in contact with first layer 104a and second electrode 106; and insulating isolation layer 104i. The degree of oxygen deficiency of second layer 104b is lower than that of first layer 104a. For example, first layer 104a is TaOₓ. Second layer 104b is Ta₂O₅ whose degree of oxygen deficiency is lower than that of first layer 104a. Moreover, metal oxide layer 104 includes insulating isolation layer 104i at the perimeter, in plan view, of first electrode 103.

Here, plan view means viewing hydrogen sensor 100 according to the present disclosure from a viewpoint in the layer-stacking direction in FIG. 2A; in other words, viewing from a viewpoint in the direction normal to any of the surfaces of, for example, first electrode 103 and second electrode 106 that are planar. For example, plan view refers to viewing the top surface of hydrogen sensor 100 illustrated in FIG. 2B.

The resistance value of metal oxide layer 104 decreases according to hydrogen-containing gas in contact with second electrode 106 (i.e., the resistance value of metal oxide layer 104 decreases as the amount of the hydrogen-containing gas increases). Specifically, when hydrogen-containing gas is present in detection-target gas, hydrogen atoms are dissociated from the hydrogen-containing gas at second electrode 106. The hydrogen atoms dissociated enter metal oxide layer 104 and form an impurity level. Particularly, the hydrogen atoms dissociated concentrate on the vicinity of the interface with second electrode 106 and decrease the apparent thickness of second layer 104b. As a result, the resistance value of metal oxide layer 104 decreases.

Second electrode 106 is a planar electrode that is capable of dissociating hydrogen and includes two surfaces. Of the two surfaces of second electrode 106, one surface (i.e., the lower surface in FIG. 2A) is in contact with metal oxide layer 104, and the other surface (i.e., the upper surface in FIG. 2A) is in contact with metal layer 106s and the outside air. Second electrode 106 includes, in opening 106a, exposed portion 106e that is exposed to the outside air. Second electrode 106 is formed of a material having a function of catalyzing dissociation of hydrogen atoms from gas molecules having the hydrogen atoms, for example: noble metal such as platinum (Pt), iridium (Ir), or palladium (Pd); or nickel (Ni); or an alloy containing at least one of these. It is assumed that second electrode 106 in FIG. 2A is platinum (Pt). Two terminals, namely first terminal TE1 and second terminal TE2, are connected to second electrode 106.

First terminal TE1 is connected to second electrode 106 through via 108.

Second terminal TE2 is connected to second electrode 106 through via 108. When hydrogen sensor 100 is used in the horizontal mode, first terminal TE1 and second terminal TE2 are connected, through openings TE1a and TE2a, to an external detection circuit (here, resistor R1 and reference element 100a) that drives hydrogen sensor 100.

As illustrated in FIG. 2B, first terminal TE1 and second terminal TE2 are arranged at positions between which exposed portion 106e is disposed in plan view of second electrode 106. Because of this arrangement, application of a predetermined voltage between first terminal TE1 and second terminal TE2 causes passage of current through exposed portion 106e of second electrode 106, that is, causes current to flow through exposed portion 106e. The passage of current through exposed portion 106e of second electrode 106 is considered to activate hydrogen dissociation by exposed portion 106e. It should be noted that the predetermined voltage may be voltages that are opposite to each other in polarity.

In hydrogen sensor 100, the resistance value between first terminal TE1 and second terminal TE2 changes when gas molecules containing hydrogen atoms come into contact with exposed portion 106e during the passage of current through exposed portion 106e. By the above-described detection circuit detecting this change in resistance value (this detection is also referred to as the “horizontal mode”), gas molecules containing low-concentration hydrogen atoms are detected.

Third terminal BE is connected to first electrode 103 through opening BEa, via 108, wiring 114, and via 108. Third terminal BE is connected, through opening BEa, to the external detection circuit that drives hydrogen sensor 100. In hydrogen sensor 100, the resistance between first electrode 103 and second electrode 106 changes when gas molecules containing hydrogen atoms come into contact with exposed portion 106e during the passage of current through exposed portion 106e. In other words, in hydrogen sensor 100, the resistance value between third terminal BE and at least one of first terminal TE1 or second terminal TE2 changes when gas molecules containing hydrogen atoms come into contact with exposed portion 106e during the passage of current through exposed portion 106e. By the above detection circuit detecting this change in resistance value (this detection is also referred to as the “vertical mode”), gas molecules containing high-concentration hydrogen atoms are detected.

It should be noted that insulating film 102, insulating films 107a to 107c, and insulating films 109a and 109b that cover main components of hydrogen sensor 100 are each formed of a silicon oxide film, a silicon nitride film, or the like.

Metal layer 106s is provided on the upper surface of second electrode 106 excluding opening 106a. Metal layer 106s is made of, for example, TiAlN, and is formed as an etching stopper for forming a via, but is not essential.

The layered structure of first electrode 103, oxide layer 104, and second electrode 106 is an element that is usable as a storage element of resistive random access memory (ReRAM). The storage element of the resistive random access memory is a digital storage element which uses, among possible states of metal oxide layer 104, two states that are a high-resistance state and a low-resistance state. Hydrogen sensor 100 according to the present disclosure uses the high-resistance state among the possible states of metal oxide layer 104. However, hydrogen detection device 10 according to the present disclosure is not limited to a configuration in which the high-resistance state is used, and may have a configuration in which the low-resistance state is used.

It should be noted that although an example in which metal oxide layer 104 has a two-layer configuration including first layer 104a made of TaOₓ and second layer 104b made of Ta₂O₅ whose degree of oxygen deficiency is low has been illustrated in FIG. 2A, metal oxide layer 104 may have a single layer configuration including a layer made of TaOₓ or Ta₂O₅ whose degree of oxygen deficiency is low.

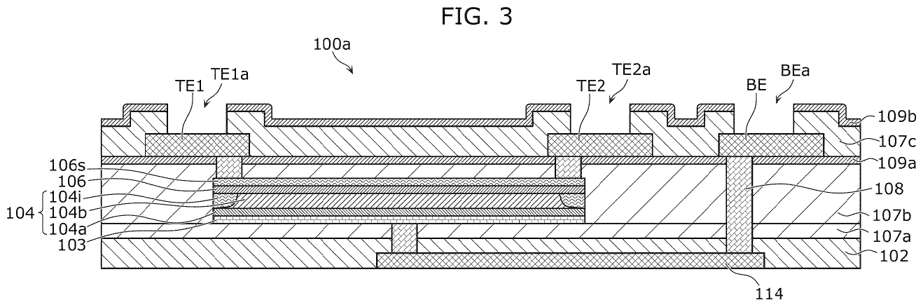

FIG. 3 is a cross-sectional view illustrating a configuration example of reference element 100a illustrated in FIG. 1.

As can be seen by comparing the present drawing and FIG. 2A, reference element 100a corresponds to one in which no opening 106a is provided (i.e., opening 106a is closed) in hydrogen sensor 100 illustrated in FIG. 2A. In other words, reference element 100a includes, as main constituent elements: a third electrode (first electrode 103 in FIG. 3) including a principal surface and a fourth electrode (second electrode 106 in FIG. 3) including a principal surface, the principal surface of the third electrode and the principal surface of the fourth electrode facing each other; a second metal oxide layer (metal oxide layer 104 in FIG. 3) disposed in contact with the principal surface of the third electrode (first electrode 103 in FIG. 3) and the principal surface of the fourth electrode (second electrode 106 in FIG. 3); and a second insulating film (insulating films 107a to 107c, 109a, and 109b in FIG. 3) that covers the third electrode (first electrode 103 in FIG. 3), the fourth electrode (second electrode 106 in FIG. 3), and the second metal oxide layer (metal oxide layer 104 in FIG. 3). The second insulating film includes no opening that is not covered by the second insulating film and through which part of an other surface of the fourth electrode (second electrode 106 in FIG. 3) opposite to the principal surface of the fourth electrode is exposed.

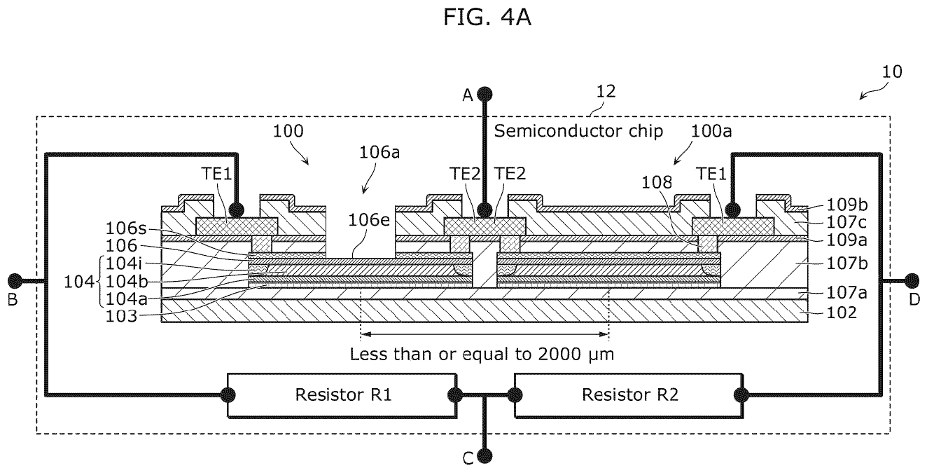

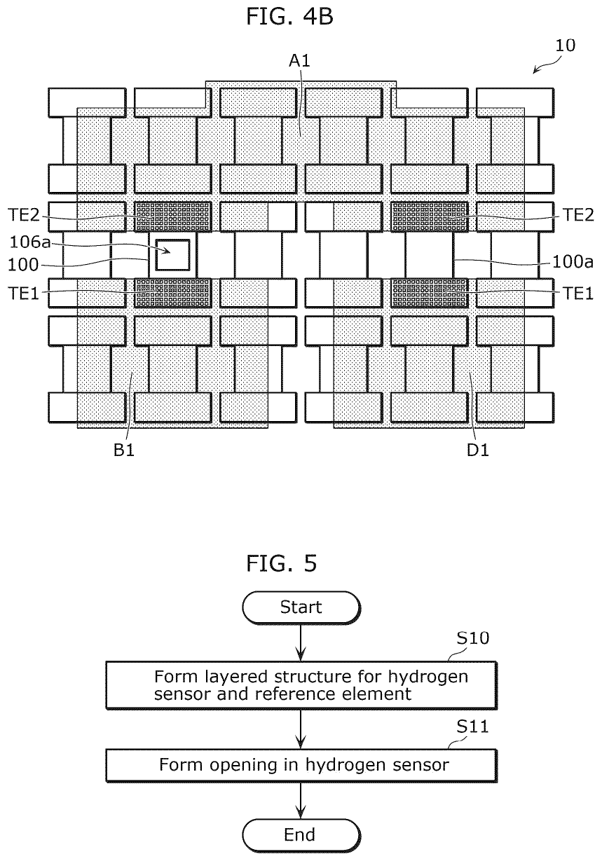

FIG. 4A is a schematic view of a configuration example of hydrogen detection device 10 according to the embodiment as a whole. Here, each of hydrogen sensor 100 and reference element 100a is dedicated to the horizontal mode (i.e., does not include third terminal BE), and the cross-sectional structure of each of hydrogen sensor 100 and reference element 100a is illustrated, whereas resistors R1 and R2 are functionally illustrated.

As illustrated in the present drawing, the feature of hydrogen detection device 10 is that four resistive elements (hydrogen sensor 100, reference element 100a, resistors R1 and R2) included in a bridge circuit are provided on a single semiconductor chip 12, and in particular, that the distance, in plan view, between hydrogen sensor 100 and reference element 100a that have basically the same configuration is less than or equal to 2000 μm.

FIG. 4B is a plan view illustrating an example of a layout of wiring patterns of hydrogen sensor 100 and reference element 100a in hydrogen detection device 10 illustrated in FIG. 4A. The present plan view illustrates: wiring pattern A1 that connects second terminal TE2 of hydrogen sensor 100 with opening 106a, second terminal TE2 of reference element 100a without an opening, and terminal A of the bridge circuit; wiring pattern B1 that connects first terminal TE1 of hydrogen sensor 100 and terminal B of the bridge circuit; and wiring pattern D1 that connects first terminal TE1 of reference element 100a and terminal D of the bridge circuit.

FIG. 5 is a flowchart illustrating a manufacturing method for manufacturing hydrogen detection device 10 according to the embodiment. Here, the manufacturing method focusing on hydrogen sensor 100 and reference element 100a among the four resistive elements included in hydrogen detection device 10 illustrated in FIG. 4A is illustrated.

Film formation and photolithography (pattern transferring and etching) that are for forming a layered structure for hydrogen sensor 100 and reference element 100a are repeatedly performed on a semiconductor substrate to form a layered structure including, from the bottom: insulating film 102 containing, for example, high density plasma fluorine doped glass (HDP-FSG); insulating film 107a as an inter-layer insulating film containing, for example, plasma tetra ethoxy silane (P-TEOS); first electrode 103 containing, for example, TaN or TIN; metal oxide layer 104 including, for example, a layered structure of Ta₂O₅ and TaO_{1.5}; second electrode 106 containing, for example, Pt; metal layer 106s containing, for example, TiAlN; insulating film 107b as an inter-layer insulating film containing, for example, P-TEOS; insulating film 109a as a protective film containing, for example, plasma silicon oxynitride film (P-SiON); first terminal TE1 and second terminal TE2 as electrodes each containing, for example, Au; insulating film 107c as an inter-layer insulating film containing, for example, high density plasma nitrogen doped glass (HDP-NSG); and insulating film 109b as a protective film containing, for example, P-SiON (layered structure forming step S10). By this step, a finished product of reference element 100a and an intermediate product of hydrogen sensor 100 in which opening 106a has not yet been formed are manufactured.

Next, photolithography (pattern transferring and etching) is performed on the intermediate product of hydrogen sensor 100 to remove, in a rectangular shape, part of metal layer 106s, insulating film 107b, insulating film 109a, insulating film 107c, and insulating film 109b so that part of the upper surface of second electrode 106 is exposed, and thus opening 106a of hydrogen sensor 100 is formed (opening forming step S11). By this step, the manufacture of hydrogen sensor 100 is completed.

Next, a result of an experiment regarding the characteristics of hydrogen detection device 10 according to the embodiment manufactured as above is described.

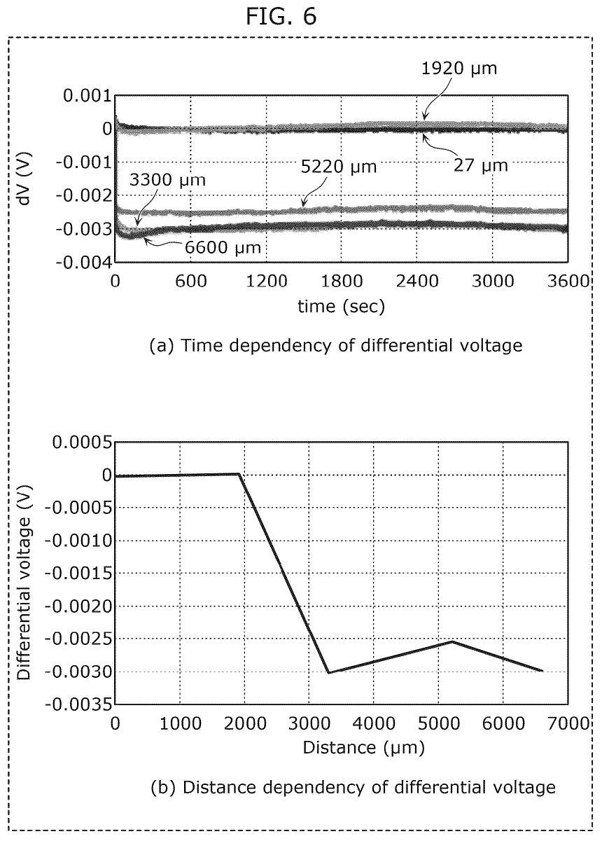

FIG. 6 is a diagram illustrating a result of an experiment regarding time dependency and distance dependency of an output voltage (differential voltage) of hydrogen detection device 10 according to the embodiment.

(a) in FIG. 6 is a diagram obtained by, in a hydrogen-free environment, changing, as a parameter, the distance, in plan view, between hydrogen sensor 100 and reference element 100a of hydrogen detection device 10 illustrated in FIG. 4A and recording time (the horizontal axis) dependency of the differential voltage (the vertical axis) between terminals B and D of the bridge circuit. Sample hydrogen detection devices 10 having respective distances of 27 μm, 1920 μm, 3300 μm, 5220 μm, and 6600 μm between hydrogen sensor 100 and reference element 100a were manufactured, and the differential voltage of each of the sample hydrogen detection devices 10 was measured.

As can be seen from the present drawing, the differential voltage of each of the sample hydrogen detection devices 10 having respective distances of 27 μm and 1920 μm was almost 0 (V) that is an ideal value, whereas the differential voltage of each of the sample hydrogen detection devices 10 having respective distances of 3300 μm, 5220 μm, and 6600 μm was a significant value (i.e., offset voltage) that is different from the ideal value. It should be noted that the differential voltage has hardly changed over time no matter which of the distances hydrogen detection device 10 has.

(b) in FIG. 6 is a diagram in which the result obtained in (a) in FIG. 6 has been rewritten as distance (the horizontal axis) dependency of the differential voltage (the vertical axis).

As can be seen from the present drawing, in a hydrogen-free environment, when the distance, in plan view, between hydrogen sensor 100 and reference element 100a included in the bridge circuit is less than or equal to about 2000 μm, the differential voltage outputted from the bridge circuit is not dependent on time and is stable and 0 (V) that is the ideal value, whereas when the distance is greater than 2000 μm, the differential voltage outputted from the bridge circuit is a significant value that is different from the ideal value. It is considered that this is because, even though hydrogen sensor 100 and reference element 100a are provided on the same semiconductor chip 12 and have basically the same structure, subtle difference in characteristics between constituent elements of hydrogen sensor 100 and constituent elements of reference element 100a due to difference in arrangement position on semiconductor chip 12 is detected by the highly sensitive bridge circuit when the distance between hydrogen sensor 100 and reference element 100a is greater than or equal to a predetermined distance.

Accordingly, it can be seen that when the distance, in plan view, between hydrogen sensor 100 and reference element 100a included in the bridge circuit is less than or equal to about 2000 μm, an offset voltage due to the distance is not generated and hydrogen can be detected stably by the highly sensitive bridge circuit.

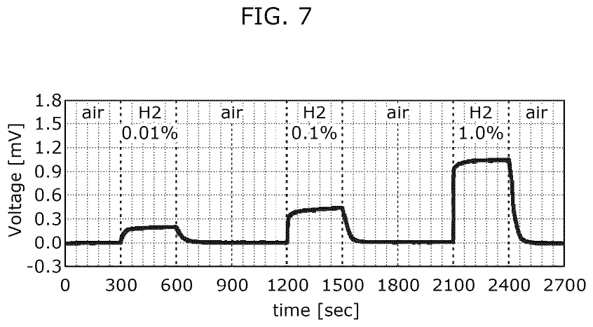

FIG. 7 is a diagram illustrating a result of an experiment regarding reaction, to hydrogen, of hydrogen detection device 10 according to the embodiment. Here, FIG. 7 shows the differential voltage (the vertical axis) outputted from hydrogen detection device 10 obtained by: confirming that the differential voltage of hydrogen detection device 10 in which the distance between hydrogen sensor 100 and reference element 100a is 27 μm is 0 mV in a hydrogen-free environment; causing the hydrogen concentration in the environment around hydrogen detection device 10 to be 0.01% for 300 msec; causing the air in the environment around hydrogen detection device 10 to be 100% (i.e., the hydrogen concentration to be 0%) for 600 msec; causing the hydrogen concentration in the environment around hydrogen detection device 10 to be 0.1% for 300 msec; causing the air in the environment around hydrogen detection device 10 to be 100% (i.e., the hydrogen concentration to be 0%) for 600 msec; causing the hydrogen concentration in the environment around hydrogen detection device 10 to be 1.0% for 300 msec; and then causing the air in the environment around hydrogen detection device 10 to be 100% (i.e., the hydrogen concentration to be 0%). It should be noted that a value of concentration is a percentage (%) by volume in gas.

As can be seen from the present drawing, hydrogen detection device 10 including hydrogen sensor 100 and reference element 100a separated from each other by a distance of 27 μm outputs a differential voltage according to change in the hydrogen concentration in the environment. Specifically, after reaction with hydrogen, when hydrogen has disappeared from the environment, the differential voltage outputted from hydrogen detection device 10 returns to the base voltage that is 0 mV and no offset voltage is generated.

It should be noted that although the distance between hydrogen sensor 100 and reference element 100a was 27 μm in the present experimental example, it is considered that the same result can be obtained as long as the distance is less than or equal to 2000 μm.

Next, a hydrogen detection device according to each variation of the embodiment is described.

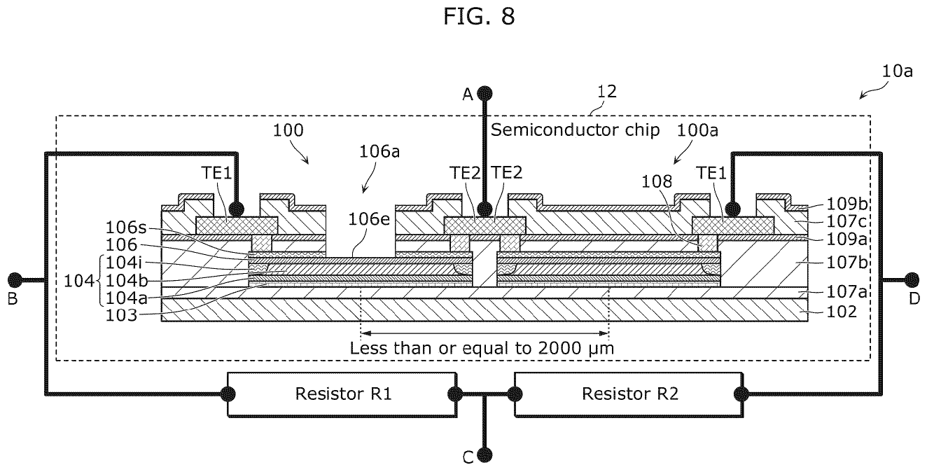

FIG. 8 is a schematic view of a configuration example of hydrogen detection device 10a according to Variation 1 of the embodiment as a whole. The difference from hydrogen detection device 10 according to the embodiment illustrated in FIG. 4A is that, in hydrogen detection device 10a according to Variation 1, among four resistive elements included in a bridge circuit, only hydrogen sensor 100 and reference element 100a are provided on a single semiconductor chip 12 and the other two among the four resistive elements, i.e., two resistors R1 and R2 are provided outside of semiconductor chip 12 (e.g., provided on a printed circuit board not illustrated).

It is considered that such hydrogen detection device 10a according to Variation 1 of the embodiment also has the same characteristics (characteristics illustrated in FIG. 6 and FIG. 7) as hydrogen detection device 10 according to the embodiment since hydrogen detection device 10a is similar to hydrogen detection device 10 according to the embodiment in that hydrogen sensor 100 and reference element 100a are provided on the single semiconductor chip 12, have basically the same structure, and are separated from each other by a distance of less than or equal to 2000 μm in plan view.

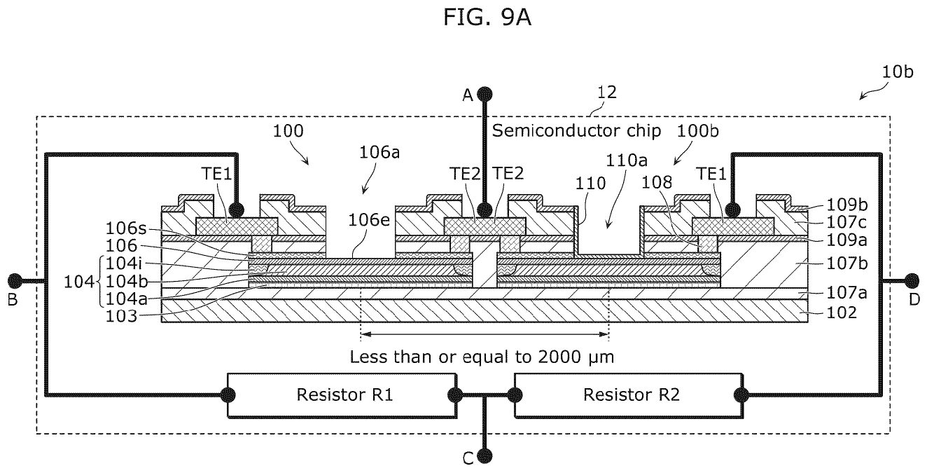

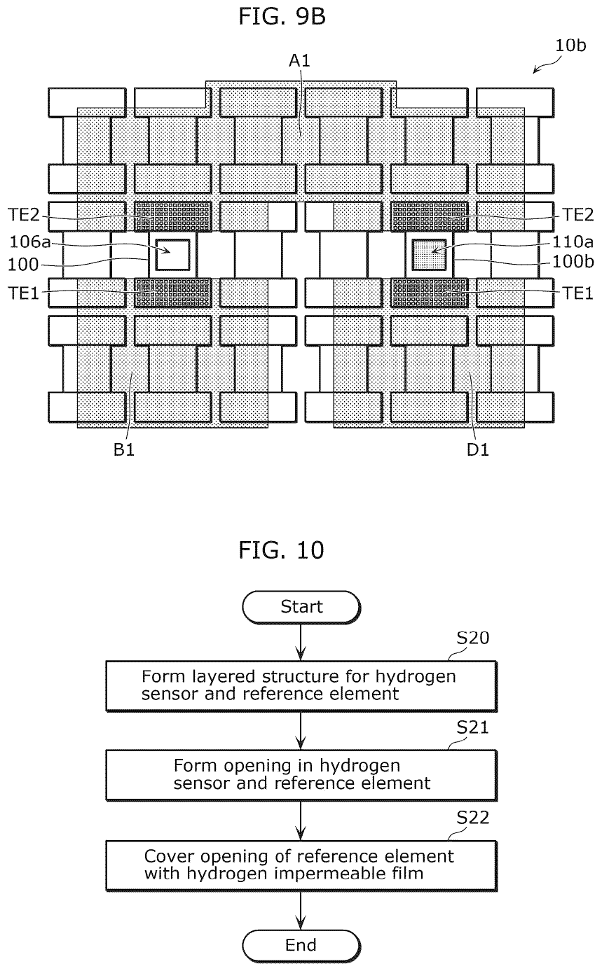

FIG. 9A is a schematic view of a configuration example of hydrogen detection device 10b according to Variation 2 of the embodiment as a whole. The difference from hydrogen detection device 10 according to the embodiment illustrated in FIG. 4A is that, in hydrogen detection device 10b according to Variation 2, in the manufacturing process, opening 110a similar to opening 106a of hydrogen sensor 100 is formed in reference element 100b and then the inner side surface and the bottom surface of opening 110a are covered by hydrogen impermeable film 110.

FIG. 9B is a plan view illustrating an example of a layout of wiring patterns of hydrogen sensor 100 and reference element 100b in hydrogen detection device 10b illustrated in FIG. 9A. The present plan view illustrates: wiring pattern A1 that connects second terminal TE2 of hydrogen sensor 100 with opening 106a, second terminal TE2 of reference element 100b with opening 110a, and terminal A of the bridge circuit; wiring pattern B1 that connects first terminal TE1 of hydrogen sensor 100 and terminal B of the bridge circuit; and wiring pattern D1 that connects first terminal TE1 of reference element 100b and terminal D of the bridge circuit.

It is considered that such hydrogen detection device 10b according to Variation 2 of the embodiment also has the same characteristics (characteristics illustrated in FIG. 6 and FIG. 7) as hydrogen detection device 10 according to the embodiment since hydrogen detection device 10b is similar to hydrogen detection device 10 according to the embodiment in that hydrogen sensor 100 and reference element 100b are provided on the single semiconductor chip 12, have basically the same structure, and are separated from each other by a distance of less than or equal to 2000 μm in plan view.

FIG. 10 is a flowchart illustrating a manufacturing method for manufacturing hydrogen detection device 10b according to Variation 2 of the embodiment. Here, the manufacturing method focusing on hydrogen sensor 100 and reference element 100b among the four resistive elements included in hydrogen detection device 10b illustrated in FIG. 9A is illustrated.

Film formation and photolithography (pattern transferring and etching) for forming a layered structure for hydrogen sensor 100 and reference element 100b are repeatedly performed on a semiconductor substrate to form a layered structure including, from the bottom: insulating film 102 containing, for example, high density plasma fluorine doped glass (HDP-FSG); insulating film 107a as an inter-layer insulating film containing, for example, plasma tetra ethoxy silane (P-TEOS); first electrode 103 containing, for example, TaN or TIN; metal oxide layer 104 including, for example, a layered structure of Ta₂O₅ and TaO_{1.5}; second electrode 106 containing, for example, Pt; metal layer 106s containing, for example, TiAlN; insulating film 107b as an inter-layer insulating film containing, for example, P-TEOS; insulating film 109a as a protective film containing, for example, plasma silicon oxynitride film (P-SiON); first terminal TE1 and second terminal TE2 as electrodes each containing, for example, Au; insulating film 107c as an inter-layer insulating film containing, for example, high density plasma nitrogen doped glass (HDP-NSG); and insulating film 109b as a protective film containing, for example, P-SiON (layered structure forming step S20). By this step, an intermediate product of hydrogen sensor 100 in which opening 106a has not yet been formed and an intermediate product of reference element 100b in which opening 110a has not yet been formed are manufactured.

Next, photolithography (pattern transferring and etching) is performed on each of the intermediate product of hydrogen sensor 100 and the intermediate product of reference element 100b to remove, in a rectangular shape, part of metal layer 106s, insulating film 107b, insulating film 109a, insulating film 107c, and insulating film 109b so that part of the upper surface of second electrode 106 is exposed, and thus opening 106a of hydrogen sensor 100 and opening 110a of reference element 100b are formed (opening forming step S21). By this step, the manufacture of hydrogen sensor 100 is completed.

Lastly, the inner side surface and the bottom surface of opening 110a formed in reference element 100b are covered by hydrogen impermeable film 110 containing, for example, P-SiON (hydrogen impermeable film forming step S22). By this step, the manufacture of reference element 100b provided with opening 110a including the inner side surface and the bottom surface that are covered by hydrogen impermeable film 110 is completed. It should be noted that a process that is the same as the process of forming insulating film 109b (i.e., film formation using the same material as insulating film 109b) may be performed for forming hydrogen impermeable film 110.

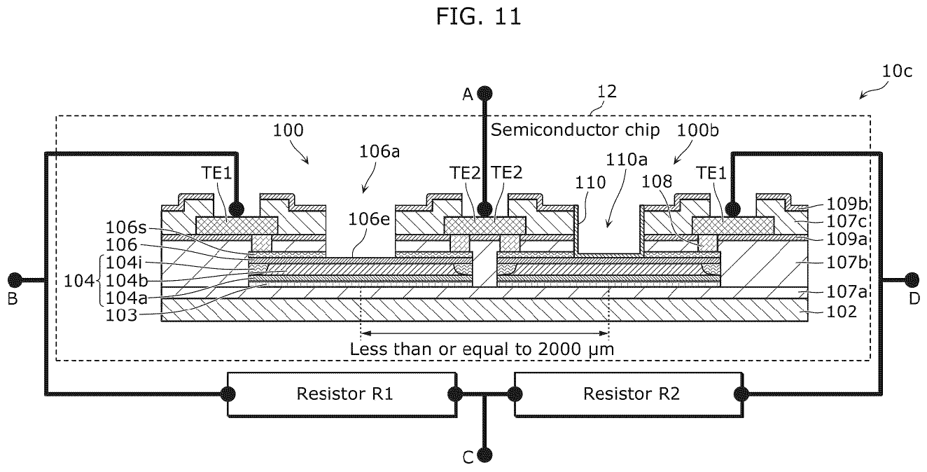

FIG. 11 is a schematic view of a configuration example of hydrogen detection device 10c according to Variation 3 of the embodiment as a whole. The difference from hydrogen detection device 10b according to Variation 2 of the embodiment illustrated in FIG. 9A is that, among four resistive elements included in a bridge circuit in hydrogen detection device 10c according to Variation 3, only hydrogen sensor 100 and reference element 100b are provided on a single semiconductor chip 12 and the other two among the four resistive elements, i.e., two resistors R1 and R2 are provided outside of semiconductor chip 12 (e.g., provided on a printed circuit board not illustrated).

It is considered that such hydrogen detection device 10c according to Variation 3 of the embodiment also has the same characteristics (characteristics illustrated in FIG. 6 and FIG. 7) as hydrogen detection device 10 according to the embodiment since hydrogen detection device 10c is similar to hydrogen detection device 10 according to the embodiment in that hydrogen sensor 100 and reference element 100b are provided on the single semiconductor chip 12, have basically the same structure, and are separated from each other by a distance of less than or equal to 2000 μm in plan view.

As described above, hydrogen detection device 10 or the like according to the embodiment includes a bridge circuit including hydrogen sensor 100 that is a first resistive element, resistor R1 that is a second resistive element, reference element 100a that is a third resistive element, and resistor R2 that is a fourth resistive element. One end of hydrogen sensor 100 and one end of resistor R1 are connected to each other, one end of reference element 100a and one end of resistor R2 are connected to each other, an other end of hydrogen sensor 100 and an other end of reference element 100a are connected to each other, and an other end of resistor R1 and an other end of resistor R2 are connected to each other. Among hydrogen sensor 100, resistor R1, reference element 100a, and resistor R2, at least hydrogen sensor 100 and reference element 100a are provided on a single semiconductor chip 12. Hydrogen sensor 100 includes: first electrode 103 including a principal surface and second electrode 106 including a principal surface, the principal surface of first electrode 103 and the principal surface of second electrode 106 facing each other; a first metal oxide layer (first metal oxide layer 104 in FIG. 2A) disposed in contact with the principal surface of first electrode 103 and the principal surface of second electrode 106; and a first insulating film (insulating films 107a to 107c, 109a, and 109b) that covers first electrode 103, second electrode 106, and the first metal oxide layer (metal oxide layer 104 in FIG. 2A). The first insulating film includes opening 106a that is not covered by the first insulating film and through which part of an other surface of second electrode 106 opposite to the principal surface of second electrode 106 is exposed. Reference element 100a includes: a third electrode (first electrode 103 in FIG. 3) including a principal surface and a fourth electrode (second electrode 106 in FIG. 3) including a principal surface, the principal surface of the third electrode and the principal surface of the fourth electrode facing each other; a second metal oxide layer (metal oxide layer 104 in FIG. 3) disposed in contact with the principal surface of the third electrode (first electrode 103 in FIG. 3) and the principal surface of the fourth electrode (second electrode 106 in FIG. 3); and a second insulating film (insulating films 107a to 107c, 109a, and 109b in FIG. 3) that covers the third electrode (first electrode 103 in FIG. 3), the fourth electrode (second electrode 106 in FIG. 3), and the second metal oxide layer (metal oxide layer 104 in FIG. 3). The second insulating film includes no opening that is not covered by the second insulating film and through which part of an other surface of the fourth electrode (second electrode 106 in FIG. 3) opposite to the principal surface of the fourth electrode is exposed.

Accordingly, since hydrogen sensor 100 and reference element 100a included in the highly sensitive bridge circuit are variable resistive elements having basically the same structure and are provided on the single semiconductor chip 12, hydrogen sensor 100 and reference element 100a indicate the resistance values quite similar to each other in a hydrogen-free environment, whereas a potential difference is generated between two connection points in a hydrogen-containing environment due to disruption of the resistance balance in the bridge circuit including hydrogen sensor 100 and reference element 100a. Thus, a hydrogen detection device that can operate stably and does not necessarily require a heater is realized.

Moreover, in plan view of second electrode 106, a distance between hydrogen sensor 100 and reference element 100a is less than or equal to 2000 μm. Accordingly, difference in characteristics between hydrogen sensor 100 and reference element 100a due to difference in arrangement position on semiconductor chip 12 is surely suppressed, and a hydrogen detection device that can surely and stably operates with high precision is realized.

Moreover, hydrogen sensor 100 includes, as the one end and the other end of hydrogen sensor 100, first terminal TE1 and second terminal TE2 that are connected, through via 108, to the other surface of second electrode 106. Accordingly, since hydrogen sensor 100 can be used in a horizontal mode that is highly sensitive, a hydrogen detection device that is suitable for detecting low-concentration hydrogen is realized.

It should be noted that in plan view of second electrode 106, opening 106a is provided between first terminal TE1 and second terminal TE2. Accordingly, opening 106a is located on a path of current, and change in resistance at opening 106a is surely detected.

It should be noted that hydrogen sensor 100 may include, as the one end and the other end of hydrogen sensor 100, a terminal (first terminal TE1 or second terminal TE2) connected, through via 108, to the other surface of second electrode 106 and third terminal BE connected, through via 108, to an other surface of first electrode 103 opposite to the principal surface of first electrode 103. Accordingly, since hydrogen sensor 100 becomes less sensitive compared to when hydrogen sensor 100 is used in the horizontal mode, a hydrogen detection device that is suitable for detecting high-concentration hydrogen is realized.

Moreover, in hydrogen detection device 10b according to Variation 2, a second insulating film in reference element 100b includes a second opening (opening 110a in FIG. 9A) at a position that corresponds to opening 106a in the first insulating film of hydrogen sensor 100, the second opening including an inner side surface and a bottom surface that are covered by hydrogen impermeable film 110. Accordingly, in manufacture of hydrogen sensor 100 and reference element 100b, a process of forming an opening can be provided in common, and thus a manufacturing process for an opening can be made common.

Moreover, hydrogen sensor 100, resistor R1, reference element 100a, and resistor R2 are provided on a single semiconductor chip 12 in hydrogen detection device 10 according to the embodiment. Accordingly, a small hydrogen detection device is realized.

Moreover, a manufacturing method for manufacturing hydrogen detection device 10 or the like according to the embodiment is a manufacturing method for manufacturing hydrogen detection device 10 that includes a bridge circuit including: hydrogen sensor 100 that is a first resistive element; resistor R1 that is a second resistive element; reference element 100a that is a third resistive element; and resistor R2 that is a fourth resistive element. The manufacturing method includes: layered structure forming step S20 of forming a layered structure for hydrogen sensor 100 and reference element 100a; opening forming step S21 of forming an opening in the layered structure formed. In layered structure forming step S20, a layered structure including: first electrode 103 including a principal surface and second electrode 106 including a principal surface, the principal surface of first electrode 103 and the principal surface of second electrode 106 facing each other; metal oxide layer 104 disposed in contact with the principal surface of first electrode 103 and the principal surface of second electrode 106; and insulating film 107b or the like that covers first electrode 103, second electrode 106, and metal oxide layer 104 is formed as a layered structure for hydrogen sensor 100 and reference element 100a. In opening forming step S21, at least a first opening (opening 106a) that is not covered by insulating film 107b or the like and through which part of an other surface of second electrode 106 opposite to the principal surface of second electrode 106 is exposed is formed in insulating film 107b or the like of a portion of the layered structure, the portion corresponding to hydrogen sensor 100.

Accordingly, since hydrogen sensor 100 and reference element 100a included in a highly sensitive bridge circuit are variable resistive elements having basically the same structure and are provided on a single semiconductor chip 12, a hydrogen detection device that does not necessarily require a heater and can operate stably is manufactured.

Moreover, in hydrogen detection device 10b or the like according to Variation 2, in opening forming step S21, in addition to the first opening (106a), a second opening (110a) that is not covered by insulating film 107b or the like and through which part of the other surface of second electrode 106 is exposed is formed in insulating film 107b or the like of an other portion of the layered structure, the other portion corresponding to reference element 100b. The manufacturing method for manufacturing the hydrogen detection device further includes hydrogen impermeable film forming step S22 of forming hydrogen impermeable film 110 that covers an inner side surface and a bottom surface of the second opening (110a) formed. Accordingly, in manufacture of hydrogen sensor 100 and reference element 100b, a process of forming an opening can be provided in common, and thus a manufacturing process for an opening can be made common.

Hereinabove, although the hydrogen detection device according to the present disclosure and the manufacturing method thereof have been described based on the embodiment and variations, the present disclosure is not limited to these embodiment and variations. Various modifications of the embodiment and variations as well as other embodiments resulting from combinations of some of the constituent elements from the embodiment and variations that may be conceived by those skilled in the art are included within the scope of the present disclosure as long as they do not depart from the essence of the present disclosure.

Although the distance between hydrogen sensor 100 and reference element 100a is less than or equal to 2000 μm in the above-described embodiment and variations, the distance is not necessarily less than or equal to 2000 μm. This is because, depending on the concentration of hydrogen to be detected, there are cases where a minute offset voltage outputted from the bridge circuit does not disturb detection of the hydrogen even when the distance between hydrogen sensor 100 and reference element 100a is greater than 2000 μm since hydrogen sensor 100 and reference element 100a having basically the same structure have quite similar characteristics when hydrogen sensor 100 and reference element 100a are provided on the same semiconductor chip 12.

Moreover, upon detection of low-concentration hydrogen and suppression of offset voltage outputted from the bridge circuit, the distance between hydrogen sensor 100 and reference element 100a may be an arbitrary distance, for example, less than or equal to 1500 μm, less than or equal to 1000 μm, less than or equal to 500 μm, less than or equal to 100 μm, less than or equal to 50 μm, less than or equal to 30 μm, or the minimum distance in the manufacturing process, as long as the distance is less than or equal to 2000 μm.

Furthermore, although an example in which only a hydrogen detection device is provided on semiconductor chip 12 has been described in each of the above-described embodiment and variations, a circuit other than the hydrogen detection device, for example, a buffer amplifier that amplifies a differential voltage outputted from the bridge circuit, a constant voltage power supply circuit that generates a voltage to be applied to the bridge circuit, or the like may also be provided.

### INDUSTRIAL APPLICABILITY

A hydrogen detection device according to the present disclosure can be used as a hydrogen detection device that operates stably with high sensitivity using a bridge circuit, for example, as a hydrogen detection device that is provided to a fuel-cell vehicle.

1. A hydrogen detection device comprising:
a bridge circuit including a first resistive element, a second resistive element, a third resistive element, and a fourth resistive element, wherein
one end of the first resistive element and one end of the second resistive element are connected to each other,
one end of the third resistive element and one end of the fourth resistive element are connected to each other,
an other end of the first resistive element and an other end of the third resistive element are connected to each other,
an other end of the second resistive element and an other end of the fourth resistive element are connected to each other,
among the first resistive element, the second resistive element, the third resistive element, and the fourth resistive element, at least the first resistive element and the third resistive element are provided on a single semiconductor chip,
the first resistive element is a hydrogen sensor and includes:
a first electrode including a principal surface and a second electrode including a principal surface, the principal surface of the first electrode and the principal surface of the second electrode facing each other;
a first metal oxide layer disposed in contact with the principal surface of the first electrode and the principal surface of the second electrode; and
a first insulating film that covers the first electrode, the second electrode, and the first metal oxide layer,
the first insulating film includes a first opening that is not covered by the first insulating film and through which part of an other surface of the second electrode opposite to the principal surface of the second electrode is exposed,
the third resistive element is a reference element and includes:
a third electrode including a principal surface and a fourth electrode including a principal surface, the principal surface of the third electrode and the principal surface of the fourth electrode facing each other;
a second metal oxide layer disposed in contact with the principal surface of the third electrode and the principal surface of the fourth electrode; and
a second insulating film that covers the third electrode, the fourth electrode, and the second metal oxide layer, and
the second insulating film includes no opening that is not covered by the second insulating film and through which part of an other surface of the fourth electrode opposite to the principal surface of the fourth electrode is exposed.

2. The hydrogen detection device according to claim 1, wherein
in plan view of the second electrode, a distance between the first resistive element and the third resistive element is less than or equal to 2000 μm.

3. The hydrogen detection device according to claim 1, wherein
the first resistive element includes, as the one end and the other end of the first resistive element, a first terminal and a second terminal that are connected, through a via, to the other surface of the second electrode.

4. The hydrogen detection device according to claim 3, wherein
in plan view of the second electrode, the first opening is provided between the first terminal and the second terminal.

5. The hydrogen detection device according to claim 1, wherein
the first resistive element includes, as the one end and the other end of the first resistive element, a terminal connected, through a via, to the other surface of the second electrode and a third terminal connected, through a via, to an other surface of the first electrode opposite to the principal surface of the first electrode.

6. The hydrogen detection device according to claim 1, wherein
the second insulating film includes a second opening at a position that corresponds to the first opening in the first insulating film, the second opening including an inner side surface and a bottom surface that are covered by a hydrogen impermeable film.

7. The hydrogen detection device according to claim 1, wherein
the first resistive element, the second resistive element, the third resistive element, and the fourth resistive element are provided on the single semiconductor chip.

8. A manufacturing method for manufacturing a hydrogen detection device that includes a bridge circuit including: a first resistive element that is a hydrogen sensor; a second resistive element; a third resistive element that is a reference element; and a fourth resistive element, the manufacturing method comprising:
forming a layered structure for the first resistive element and the third resistive element; and
forming an opening in the layered structure formed, wherein
in the forming of a layered structure, a layered structure including: a first electrode including a principal surface and a second electrode including a principal surface, the principal surface of the first electrode and the principal surface of the second electrode facing each other; a metal oxide layer disposed in contact with the principal surface of the first electrode and the principal surface of the second electrode; and an insulating film that covers the first electrode, the second electrode, and the metal oxide layer is formed as the layered structure for the first resistive element and the third resistive element, and
in the forming of an opening, at least a first opening that is not covered by the insulating film and through which part of an other surface of the second electrode opposite to the principal surface of the second electrode is exposed is formed in the insulating film of a portion of the layered structure, the portion corresponding to the first resistive element.

9. The manufacturing method according to claim 8, wherein
in the forming of an opening, in addition to the first opening, a second opening that is not covered by the insulating film and through which part of the other surface of the second electrode is exposed is formed in the insulating film of an other portion of the layered structure, the other portion corresponding to the third resistive element,
the manufacturing method further comprising:
forming a hydrogen impermeable film that covers an inner side surface and a bottom surface of the second opening formed.
